(19) United States
(12) Patent Application Publication
(10) Pub. No.: US 2007/0025976 A1
(43) Pub. Date: Feb. 1, 2007

(54) Process for the purification of coenzyme Q10

(75) Inventors: Michael D. Kluetz, Watertown, MN (US); Kevin D. Uptain, Minnetonka, MN (US); Timothy Oolman, Plymouth, MN (US); Alexander Patist, Minnetonka, MN (US)

Correspondence Address: CARGILL, INCORPORATED, LAW/24, 15407 MCGINTY ROAD WEST, WAYZATA, MN, 55391, US

(73) Assignee: Cargill, Inc.

(21) Appl. No.: 11/193,638

(22) Filed: Aug. 1, 2005

(51) Int. Cl. C07C 50/28

(52) U.S. Cl. 424/94.1; 552/307

(57) ABSTRACT

The present invention relates to methods for purifying Coenzyme Q₁₀.

The present invention relates to methods of purifying coenzyme Q₁₀ (hereafter “CoQ₁₀”). As used herein, CoQ₁₀ refers to 2,3 dimethoxy-5 methyl-6 decaprenyl benzoquinone, also known as ubidecarenone.

### BRIEF DESCRIPTION OF THE DRAWINGS

FIG. 1 illustrates a counter-current extraction scheme.

FIG. 2 is a graph showing the effect of water concentration on CoQ₁₀ extraction.

FIG. 3 is a graph illustrating extraction efficiency as a function of solvent composition.

FIG. 4 is a graph illustrating solubility of CoQ₁₀ as a function of solvent composition.

One aspect of the present invention relates to a method of purifying CoQ₁₀, which comprises:

(i) contacting a CoQ₁₀-containing biomass with a first solvent to yield a substantially dewatered biomass, wherein the solvent and the biomass are of a relative weight ratio that enables the solvent to adsorb water from the biomass while extracting <5% of the CoQ₁₀ contained in said biomass; and

(ii) contacting the substantially dewatered biomass with a second solvent to yield a crude CoQ₁₀ extract and a spent biomass.

Step (i) serves to wash impurities and water from the biomass, while step (ii) serves to extract CoQ₁₀ from the substantially dewatered biomass. Step (ii) may be repeated one or more times to increase the yield of the CoQ₁₀ extract.

In some embodiments of the present invention, the method further comprises:

(iii) contacting the spent biomass with water to remove entrained solvent from said spent biomass.

Any or all of steps (i), (ii) and (iii) may be carried out at a temperature below the boiling point of the solvents. In some embodiments, any or all of steps (i), (ii) and (iii) is/are carried out between room temperature and about 50° C. In other embodiments, any or all of steps (i), (ii) and (iii) is/are carried out at about 50° C.

In some embodiments, the first solvent and/or second solvent comprises about 95% w/w or more acetone in water. In other embodiments, the first and second solvents are independently selected from ethanol [EtOH] and methanol [MeOH]. In yet other embodiments, the second solvent is the same as the first solvent.

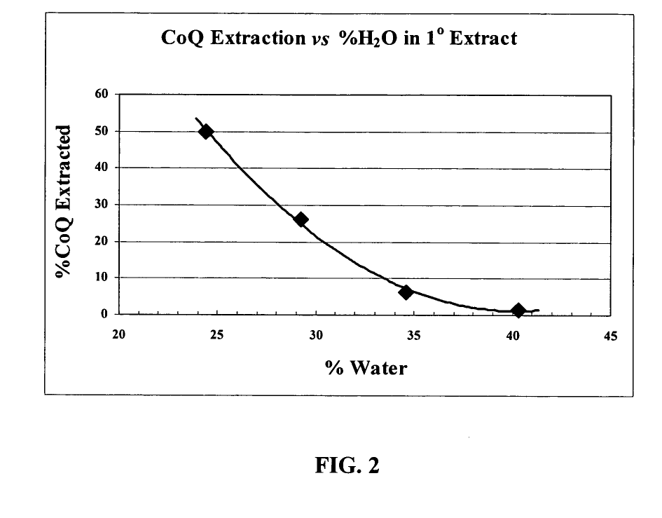

Extraction performance is sensitive to the weight percent water in the liquid portion of the extraction milieu. FIG. 2 illustrates the impact of water concentration on extraction efficiency in step (i). If the weight percent water of step (i)—measured as the weight of the combined water contributions of the biomass and the first solvent, divided by the total weight of the liquid portion (water plus organic solvent)—drops too low, then CoQ₁₀ may extract along with the impurities. Thus, in some embodiments, the weight percent water of the biomass and the first solvent are adjusted such that step (i) yields a liquid portion (that is, a wash containing predominantly impurities) comprising about 40% w/w or more water. In such embodiments, the liquid portion is substantially CoQ₁₀-free, i.e., contains about 5% or less CoQ₁₀. In other embodiments, if a substantially water-free solvent is used in step (i), the solvent to biomass dry matter weight-to-weight ratio is maintained at about 6 to 1 based on a typical biomass water concentration of about 80% w/w, i.e., the biomass cells contain about 20% w/w dry matter in step (i). A “substantially water-free” solvent refers to a solvent with less than about 1% w/w water.

If the biomass cells contain >20% w/w dry matter in step (i), one or more of the following steps may be taken to achieve a 40% w/w or more weight percent water in the step (i) wash: (a) supplement the CoQ₁₀-containing biomass cells with added water, (b) use a lower w/w [weight to weight] solvent to biomass d.b. [dry basis] ratio, and (c) use in place of the substantially water-free solvent, a solvent with a higher weight percent water, e.g., a reclaimed solvent stream that contains several weight percent water. By using a reclaimed solvent, the methods of the present invention could avoid costly distillation with rectification or ternary distillation. Thus, in some embodiments, the CoQ₁₀-containing biomass has a weight percent water of less than 80% w/w. In other embodiments, the relative weight ratio of the solvent to biomass d.b. is less than 6 to 1, or the solvent has a weight percent water of more than 1% w/w.

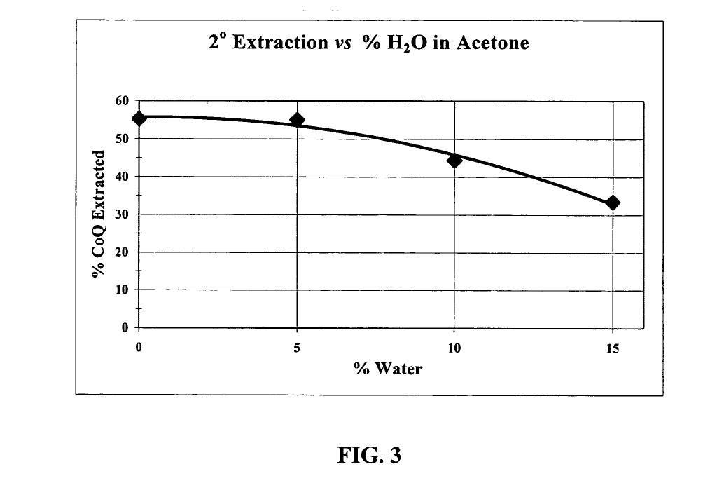

The weight percent water of solvents also affects extraction efficiency in later stages. FIG. 3 shows that CoQ₁₀ extraction efficiency drops with increasing water concentration in the solvent in step (ii). However, 5% water in acetone performs essentially the same as dry acetone. Accordingly, in some embodiments, the second solvent in step (ii) comprises about 95% w/w or more acetone in water.

Acetone may be separated from water using any method known in the art. For example, a multi-stage distillation column may produce a distillate containing about >94% w/w acetone and a bottoms stream containing wastewater with less than about 1000 ppm acetone. Such distillate is suitable for direct recycle to the extraction process.

The methods of the present invention may utilize counter-current extraction. Thus, in some embodiments, the first solvent is contacted in a cross-current manner with the CoQ₁₀-containing biomass in step (i), while the second solvent is contacted in a counter-current manner with the washed biomass in several sequential applications of step (ii). In yet other embodiments, the spent biomass is contacted in a counter-current manner with water in step (iii).

The crude extract generated by the counter-current extraction process may contain CoQ₁₀ at a concentration of about 0.1% to about 0.15% by weight. The recovery of CoQ₁₀ from this dilute extract may be accomplished by stripping out the solvent (increasing the water to solvent ratio) until an oily precipitate forms. Accordingly, in some embodiments, the method further comprises:

(iv) stripping out the solvent from the crude CoQ₁₀ extract until a precipitate forms.

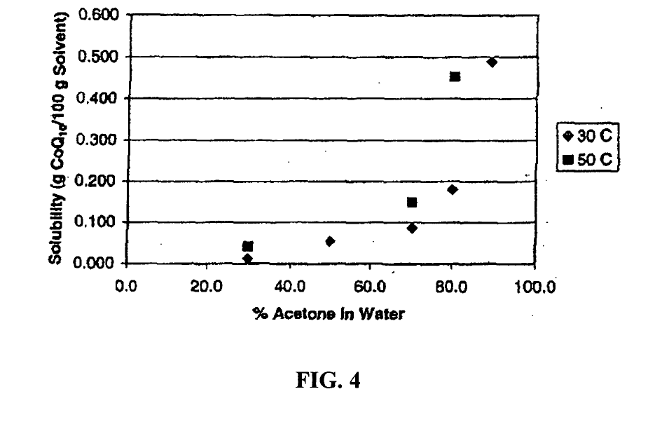

The solubility of CoQ₁₀ and other lipophilic compounds may become very low as the acetone concentration decreases below 50% in water. FIG. 4 illustrates this effect.

In some embodiments, the method further comprises:

(v) separating the precipitate from the solvent.

In these embodiments, a centrifuge may be used to separate the oily precipitate from the solvent.

In further embodiments, the method further comprises:

(vi) optionally drying the precipitate; and

(vii) chromatographing the precipitate.

In yet further embodiments, the precipitate is chromatographed on a silica gel column. The precipitate may be dissolved in hexane before loading onto the chromatography column. Silica gel chromatography can purify the CoQ₁₀ precipitate to greater than 85% purity, with a recovery of 95-100%.

In yet further embodiments, the method further comprises:

(viii) eluting CoQ₁₀ from the column with an agent comprising hexane and acetone at a volume-to-volume ratio of about 98 to 2.

Both loading and elution may be run at ambient temperatures.

The silica gel can be regenerated using an EtOH wash at ambient temperature. Warm hexane (about 50° C.) may be used to wash (i.e. desorb) residual EtOH from the system. After cool-down, the column can be loaded again with precipitate in hexane. Impurities can be removed from the EtOH stream by carbon adsorption.

Chromatography creates a dilute stream containing CoQ₁₀ in a 98:2 mixture of hexane and acetone. To prepare the CoQ₁₀ for cooling crystallization in ethanol, all hexane and acetone must be thoroughly removed. Thus, in yet further embodiments, the method further comprises:

(ix) removing the hexane and the acetone from the eluted CoQ₁₀ to yield a solvent-free CoQ₁₀ product.

One approach is to evaporate the hexane at a temperature slightly above the melting point of CoQ₁₀ (48° C.). With CoQ₁₀ in liquid state, a staged evaporation system may be used to thoroughly remove solvent. The first stage would concentrate the chromatography product stream from <1% d.s. to >45% d.s. using a simple evaporator design, e.g., falling-film or rising-film evaporator at atmospheric pressure or slight vacuum. The second stage would remove the last traces of hexane at low vacuum (e.g. 25 mm Hg), and is likely to require assisted mass transfer (e.g. wiped-film evaporator).

In yet further embodiments, the method further comprises:

(x) crystallizing the solvent-free CoQ₁₀ product.

CoQ₁₀ can be purified to approximately 99% purity by cooling crystallization in ethanol.

The methods of the present invention may be carried out without requiring any disruption of the biomass, drying of the biomass, and/or washing of fermentation broth impurities from the biomass prior to extraction.

It will be apparent to one of ordinary skill in the art that specific embodiments of the present invention may be directed to one, some or all of the above-indicated aspects as well as other aspects, and may encompass one, some or all of the above- and below-indicated embodiments, as well as other embodiments.

Other than in the working examples, or where otherwise indicated, all numbers expressing quantities of ingredients, reaction conditions, and so forth used in the specification and claims are to be understood as being modified by the term “about”. Accordingly, unless indicated to the contrary, such numbers are approximations that may vary depending upon the-desired properties sought to be obtained by the present invention. At the very least, and not as an attempt to limit the application of the doctrine of equivalents to the scope of the claims, each numerical parameter should be construed in light of the number of significant digits and ordinary rounding techniques.

While the numerical ranges and parameters setting forth the broad scope of the invention are approximations, the numerical values set forth in the working examples are reported as precisely as possible. Any numerical value, however, inherently contains certain errors necessarily resulting from the standard deviation found in their respective testing measurements.

### EXAMPLES

### Example 1

### Counter-Current Extraction Scheme

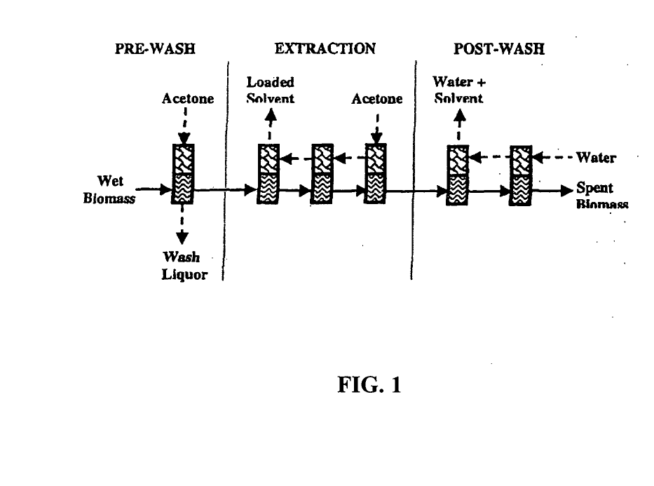

A method of the present invention is performed using a counter-current extraction scheme similar to the one illustrated in FIG. 1. The first stage is operated in a cross-current fashion to wash impurities and water from the biomass. This wash stream contains negligible CoQ₁₀ and can be sent directly to the solvent recovery system.

Acetone is then contacted counter-currently with the biomass in three stages of extraction. The solvent to biomass dry matter ratio is maintained at 6 to 1 since essentially water-free acetone is used. The biomass pellets are expected to carry approximately 65% solvent by weight with them when they are transferred from stage to stage.

Next, water is contacted counter-currently with the biomass in two stages of washing to remove entrained solvent. Each extraction stage is provided at least 30 minutes of contact time with agitation. All wash and extraction stages are operated at 50° C. Using this extraction sequence, over 95% recovery of CoQ₁₀ at an extract purity of >20% CoQ₁₀ on a dry basis may be achieved.

### Examples 2-6

Materials and Methods

The biomass used in these Examples comprised any number of variants of Rhodobacter spheroides that had been genetically manipulated. For the most part, all strain variants used in extraction had the carotenoid genes eliminated. The strains did vary in other respects, often manifested in different colors due to the presence, or absence, of other pigments such as bacterial chlorophyll, hemes and cytochromes.

Biomass was collected by centrifugation from fermentation broth. Typical levels were 5 to 25 gm/L broth [gm of dry cell weight]. The biomass pellets were used without washing. The moisture content of the pellets varied from 74.5% up to 82% [i.e. the dry cell content was 25.5% to 18%]. Dried biomass was obtained by vacuum oven drying of a small portion of wet cell mass, and was assayed to show that CoQ₁₀ was not lost in the drying process.

Extraction trials are referred to as either “mini extractions” or regular extractions in stirred cells. In “mini extractions” about 5-10 gm of wet biomass was placed in a 50 mL centrifuge tube, solvent added, and the mixture manually agitated periodically by shaking. Regular stirred cell extractions were performed in Wheaton Celstir® bioreactors of 1 L total volume. In multi-stage extractions, the biomass suspension would be removed to centrifuge bottles, the pellet spun down, removed from the bottles and placed back into the Wheaton vessel with fresh solvent for the next stage. Total solids mass balances indicated that in a 5-stage extraction series, only about 5% of the total dry matter was lost despite the repeated transfers. Unless otherwise indicated, extractions were done with a 6:1 [w:w] ratio of solvent to biomass dry matter. Hence, a typical charge to the extractor—if the biomass contained 80% moisture—would be 35 gm d.b. biomass, or 175 gm as is basis, plus 210 gm [about 265 mL, since ρₛₒₗᵥₑₙₜ˜0.8 gm/mL for most of the solvents] of solvent. This resulted in a total initial volume of about 440 mL. Regular extractions were typically done with three such Wheaton vessels in series on a common circulating water bath at 50° C.

Analysis of CoQ₁₀ was performed on a Waters HPLC system using a Waters 3.9×150 mm Nova-Pak® C₁₈ column; flow rate was 1.0 mL/min of mobile phase comprising 70% EtOH:30% MeOH [isocratic]; detection was by UV at 275 nm; injection volume was typically 20 μL. Samples were typically prepared by rotovapping to dryness 5 mL of extract to which 15-20 mL pure EtOH had been added, then redissolving the residue in 10 mL of EtOH containing 5% hexane. Care was taken to assure that all of the residue was thoroughly resuspended and all soluble components totally dissolved. Sonication can assist in this redissolution. For extracts that were originally high in water [>10%] this problem was exacerbated; in these cases 0.5 mL hexane was added separately to the flask first to redissolve the residue, followed by sonication, and finally addition of 9.5 mL EtOH.

### Example 2

### Extraction of Pre-Dried Biomass with Acetone, EtOH and Hexane

Extractions were done on dry biomass containing 6600 ppm CoQ₁₀. Experiments were designed to obtain sufficient material to agitate in stirred extractors using 12:1 solvent:dry biomass. Two stages of extraction were performed at 50° C. Results are set forth below in Table I.

TABLE I

| | Hexane | Acetone | EtOH |
|---|---|---|---|
| % Yield Stage 1 | 44 | 66 | 74 |
| % Yield Stage 2 | 14 | 12 | 15 |
| Total % Yield | 58 | 78 | 89 |
| % Purity of CoQ₁₀ | 24 | 21 | 5 |

The less polar solvents, which are known to dissolve CoQ₁₀ readily, yielded less CoQ₁₀, but at higher purity. In fact, hexane gave the worst recovery in two stages—only 58%. On the other hand, EtOH gave the highest recovery, but at very low purity due to the concomitant extraction of other cell components.

The results suggest that the presence of water, or at least the use of a more “water-like” organic solvent, is necessary to efficiently extract the CoQ₁₀, and the intrinsic solubility of CoQ₁₀ in the particular solvent is of less importance. Compared with extractions of wet biomass (see below), the extractions of dry biomass do not appear any more efficient. Thus, the methods of the present invention do not need the energy-intensive step of pre-drying to achieve efficient extractions.

### Example 3

### Three-Stage Extraction of Wet Biomass with Acetone and EtOH

Extractions were first done with acetone and EtOH at a ratio of 12:1 [w:w] in three stages at 50° C. Starting biomass had ˜5000 ppm CoQ₁₀. 12 gm d.b. biomass plus 144 gm acetone were placed in each of three stirred cells; a similar triplicate set was done with EtOH. In each stage, aliquots were withdrawn to estimate the time course of the extraction. While analytical difficulties with the acetone time course were encountered, the EtOH run indicated that extraction was complete in each stage by 30 minutes; this would later be established as the default time-of-contact per stage.

The results are as follows: acetone gave a total yield of 112%, compared to 80% for EtOH. Acetone extracted a fair amount of CoQ₁₀ in stage 1 [35%], and 66% in stage 2. EtOH only removed 5% in stage 1 and 63% in stage 2. Stage 2 in both cases gave the maximum purity CoQ₁₀, which was nearly 24% for acetone, but only 5% for EtOH. By contrast, acetone stage 1 purity was only 2.5% as the high CoQ₁₀ extractability was accompanied by an even greater extractability of large amounts of contaminants. From extract densities one can estimate the water concentration of the solutions, which was 30% and 8% for acetone stages 1 and 2, respectively, and 32% and 12% for ethanol stages 1 and 2, respectively.

### Example 4

### Comparison of Extractions of Wet Biomass with Various Alcohols

Extractions identical to those in Example 3 were done with EtOH again [as a control] as well as n-butanol [n-BuOH], isopropyl alcohol [IPA] and MeOH. In this case, the biomass had <5000 ppm CoQ₁₀. For these runs, 60 minutes per each of the three stages were allowed to insure completeness.

These results are set forth below in Table II.

TABLE II

| | EtOH | n-BuOH | IPA | MeOH |
|---|---|---|---|---|
| % Yield-1° | 4 | 63 | 73 | 0 |
| % Yield-2° | 60 | 22 | 11 | 57 |
| % Yield-Total | 75 | 87 | 85 | 89 |
| % Purity | 6.5 | 2.5 | 2 | 3.5 |

All alcohols give much lower purity than acetone. IPA and BuOH extracted the majority of the CoQ₁₀ in stage 1 at very low purity. IPA, BuOH and EtOH form azeotropes, increasing reclaim costs. MeOH extracted CoQ₁₀ and impurities in stages 2 and 3, with very low purity.

### Example 5

### Five-Stage Extractions of Wet Biomass with EtOH and Acetone

The extractions in this section were all done at a solvent:biomass ratio of 6:1. As a result, both acetone and EtOH extracted a fair amount of material—but little CoQ₁₀—in stage 1.

Since it was hypothesized that stage 1 was merely serving to wash water out of the cells, it was believed that this stage could be done quickly and at room temperature, rather than for 30 minutes at 50° C. When this experiment was done [with acetone as solvent] it was found that the extractability in stage 2 dropped considerably, with concomitant drops in percent yield of stages 3 to 5 as well and reduced purity in all stages. The net result was a total yield of <50%.

A small side experiment of “mini extractions” was performed in which 5 gm d.b. biomass 30 gm solvent was contacted (a) for 5 minutes at room temperature, (b) for 30 minutes at room temperature, and (c) for 30 minutes at 50° C. After recovering and assaying stage 1 extracts, a second stage extraction was done on all three pellets under typical conditions [30 minutes at 50° C.]. The total CoQ₁₀ extracted in stage 1 was only 2% in all three cases, but that extracted in stage 2 was (a) 35%, (b) 42% and (c) 55%. Thus, how one does stage 1 in terms of time and temperature affects how efficiently stage 2 performs. In subsequent experiments, stage 1 extraction was performed for the full 30 minutes. at elevated temperature.

Results from three EtOH extraction sets [starting CoQ₁₀=4500-9000 ppm] and five acetone sets [starting CoQ₁₀=5300 ppm] are set forth below in Tables III and IV.

TABLE III
Summary of EtOH Extractions

| Stage of | % H₂O in | % of B.M. | % of CoQ₁₀ | % Purity of |
|---|---|---|---|---|
| Extraction | Extract | Extracted | Extracted | CoQ₁₀ |
| 1° | 37 | 8.6 | 2 | 0.1 |
| 2° | 8 | 6.6 | 73 | 7.6 |
| 3° | 2 | 2.0 | 20 | 6.9 |
| 4° | 0.5 | 0.8 | 5 | 3.9 |
| 5° | 0.2 | 0.4 | 1 | 1.8 |
| Total | — | 18.4% | 101% | — |

The weighted average purity of stage 2° and 3° extracts is 7.9%.

TABLE IV
Summary of Acetone Extractions

| Stage of | % H₂O in | % of B.M. | % of CoQ₁₀ | % Purity of |
|---|---|---|---|---|
| Extraction | Extract | Extracted | Extracted | CoQ₁₀ |
| 1° | 38 | 4.6 | 5 | 0.6 |
| 2° | 6 | 1.6 | 72 | 23.6 |
| 3° | 2 | 0.4 | 15 | 18.6 |
| 4° | 1 | 0.2 | 3 | 7.0 |
| 5° | 0.2 | 0.2 | 1 | 2.1 |
| Total | — | 7.2% | 96% | — |

The weighted average purity of stage 20 and 30 extracts is 22.0%. This purity is 2.8 times that of the stage 2 and 3 EtOH extracts.

The results show that both acetone and EtOH are capable of extracting CoQ₁₀ in high yield. With lower solvent-to-biomass, most of the extractable impurities are removed in stage 1, but little to no CoQ₁₀. Most of the CoQ₁₀ is extracted in stage 2. Due to a large amount of liquid trapped in the biomass pellets, most of the remaining CoQ₁₀ is likely already “extracted”, but merely entrained in the biomass, to be washed out in stages 3-5. The water concentration of the five extracts drops from 38% in stage 1 to 6%, 2%, 1%, and finally to 0.2% in stage 5. Extracts in stages 2 and 3 typically contain the highest purity CoQ₁₀.

### Example 6

### Effect of Water Concentration on Stages 1 and 2 Extraction

A study was done in which the water concentration in the system was adjusted so that the primary extract would come out at 24 to 40% water concentration. FIG. 2 shows the results of % CoQ₁₀ extracted in stage 1 when the water concentration of that extract was 24.4%, 29.2%, 34.6% and 40.3%.

Another study was done to determine the effect of water-in-acetone on stage 2 efficiency. The biomass pellet from a stage 1 extraction that had produced a primary extract containing 40% w/w water, was subsequently extracted [still at a 6:1 solvent:pellet dry matter ratio] with 0%, 5%, 10% and 15% w/w water in the acetone. FIG. 3 illustrates the results of these four extractions. The results show that at least 5% water in the solvent can be tolerated without significantly reducing the efficiency of this stage.

### Example 7

### Precipitation

A single-stage, flash evaporator is used to concentrate the crude CoQ₁₀ extract from about 10% water to >40% water. With no rectification, the evaporator may produce an overhead stream of approximately 94% acetone in water, which is suitable for direct recycle to the extraction process. The bottoms stream containing about 40% water is further diluted to 50% w/w water and then cooled to 25° C. A centrifuge is then used to separate the resulting oily precipitate from the solvent. The oily precipitate is expected to have a purity greater than 30% CoQ₁₀. This oil is readily soluble in the hexane solvent used for chromatography (70 gm/100 gm). It may be necessary to dry the oil before sending it to chromatography to avoid contamination of the chromatography system with any entrained polar solvents. One approach to drying that could be used is wiped-film evaporation.

### Example 8

### Chromatography

The precipitate from Example 7 is subjected to silica gel column chromatography under operating conditions as set forth in Table V below.

TABLE V

| | |
|---|---|
| Resin | Silica (commercial grade) |
| | Grace Davisil ®; 60-200 mesh; pore size 150 Å; |
| | grade 62 |
| Loading | CoQ₁₀ precipitate in hexane, 5-10 g crude |
| | precipitate/100 g resin |
| Elution Solvent | Hexane:Acetone (98:2 w/w), 2 bed volumes [BV] |
| Loading and Elution | Ambient |
| Temperature | |
| Regeneration | EtOH (1 BV at ambient temperature), followed by |
| | hexane (4-5 BV at 50° C.) |
| Regeneration Solvent | Carbon adsorption |
| Cleanup | |

Chromatography creates a dilute stream containing CoQ₁₀ in a 98:2 w/w mixture of hexane and acetone, respectively. To prepare the CoQ₁₀ for cooling crystallization in ethanol, all hexane and acetone must be thoroughly removed. A relatively simple and inexpensive approach is to evaporate the hexane at a temperature slightly above the melting point of CoQ₁₀ (48° C.). With CoQ₁₀ in a liquid state, it should be possible to thoroughly remove solvent using a staged evaporation system. The first stage concentrates the chromatography product stream from <1% d.s. to >45% d.s. using a simple evaporator design (e.g. falling-film or rising-film evaporator at atmospheric pressure or slight vacuum). The second stage removes the last traces of hexane at low vacuum (e.g. 25 mm Hg) and may require assisted mass transfer (e.g. wiped-film evaporator).

### Example 9

### Crystallization

The chromatography product from Example 8 is dissolved in 200 proof ethanol to create a 9 wt-% solution at 55° C. This should create a slightly sub-saturated solution. Table VI below provides solubility data for pure CoQ₁₀ in ethanol.

TABLE VI

| Temperature | Solubility |
|---|---|
| ° C. | g/100 g Solvent |
| 30 | 0.38 |
| 50 | 8.99 |
| 60 | 11.0 |

A cooling cycle is then operated from 55° C. to 25° C. over a 16 hour period. The crystals are collected using either a filter or centrifuge and washed with cold ethanol. The ethanol mother liquor and wash streams can be cleaned by either carbon adsorption or solvent evaporation and then recycled.

The invention being thus described, it will be apparent to those skilled in the art that the same may be varied in many ways without departing from the spirit and scope of the invention. Such variations are included within the scope of the invention to be claimed.

We claim:

1. A method of purifying CoQ₁₀, which comprises:
(i) contacting a CoQ₁₀-containing biomass with a first solvent to yield a substantially dewatered biomass and an essentially CoQ₁₀-free wash, wherein the solvent and the biomass are of a relative weight ratio that enables the solvent to adsorb water from the biomass while extracting about 5% or less CoQ₁₀ from said biomass; and
(ii) contacting the substantially dewatered biomass with a second solvent to yield a crude CoQ₁₀ extract and spent biomass.

2. The method of claim 1, wherein the second solvent comprises about 95% w/w or more acetone in water.

3. The method of claim 1, wherein, the second solvent is the same as the first solvent.

4. The method of claim 1, wherein the first solvent and the second solvent are independently selected from EtOH and MeOH.

5. The method of claim 1, wherein step (ii) is repeated one or more times.

6. The method of claim 1, wherein the essentially CoQ₁₀-free wash comprises about 40% w/w or more water.

7. The method of claim 1, wherein the CoQ₁₀-containing biomass has a weight percent water of about 80% w/w.

8. The method of claim 7, wherein the first solvent is substantially water-free and the relative weight ratio of the solvent to biomass d.b. is about 6 to 1.

9. The method of claim 1, wherein the CoQ₁₀-containing biomass has a weight percent water of less than 80% w/w.

10. The method of claim 9, wherein the relative weight ratio of the solvent to biomass d.b. is less than 6 to 1, or the solvent has a weight percent water of more than 1% w/w.

11. The method of claim 1, wherein steps (i) and (ii) are carried out at a temperature from room temperature to about 50° C.

12. The method of claim 1, which further comprises:
(iii) contacting the spent biomass with water to remove entrained solvent from said spent biomass.

13. The method of claim 1, wherein the first solvent is contacted in a cross-current manner with the CoQ₁₀-containing biomass in step (i), while the second solvent is contacted in a counter-current manner with the substantially dewatered biomass in step (ii).

14. The method of claim 12, wherein the spent biomass is contacted in a counter-currently manner with water.

15. The method of claim 12, which further comprises:
(iv) stripping out the solvent from the crude CoQ₁₀ extract until a precipitate forms.

16. The method of claim 13, which further comprises:
(v) separating the precipitate from the solvent.

17. The method of claim 15, wherein a centrifuge is used in step (v).

18. The method of claim 15, which further comprises:
(vi) optionally drying the precipitate; and
(vii) chromatographing the precipitate.

19. The method of claim 18, wherein the precipitate is chromatographed on a silica gel column.

20. The method of claim 18, wherein step (vii) is carried out at ambient temperatures.

21. The method of claim 19, further comprising:
(viii) eluting CoQ₁₀ from the column with an agent comprising hexane and acetone at a weight-to-weight ratio of about 98 to 2.

22. The method of claim 21, which further comprises:
(ix) removing the hexane and the acetone from the eluted CoQ₁₀ to yield a solvent-free CoQ₁₀ product.

23. The method claim 22, which further comprises:
(x) crystallizing the solvent-free CoQ₁₀ product.
